(19) United States
(12) Patent Application Publication
(10) Pub. No.: US 2008/0275491 A1
(43) Pub. Date: Nov. 6, 2008

(54) REMOVABLE HELICAL MEDICAL FILTER

(76) Inventors: Volker Niermann, Bound Brook, NJ (US); James A. Fleming, Bethlehem, PA (US)

Correspondence Address: RONALD L. HOFER, 122 LINDBERGH LANE, MOORESVILLE, NC, 28117, US

(21) Appl. No.: 11/742,722

(22) Filed: May 1, 2007

(51) Int. Cl. A61M 29/00

(52) U.S. Cl. 606/200

(57) ABSTRACT

A medical filter intended for placement inside a tubular body passage such as a blood vessel of a patient, the medical filter having a plurality of arms radiating from an elongated spine in a helical pattern.

### BACKGROUND AND SUMMARY OF THE INVENTION

The present invention relates to medical filters which are intended to be placed inside a blood vessel or other body passage for the purpose of intercepting thrombus or particles.

Medical filters, including vena cava filters, are emplaced inside blood vessels or other body passages to trap unwanted particles. When used in a blood vessel, a medical filter should be effective to entrap thrombus, clots or other dangerous coagulations or particulate matter in the blood while allowing free flow of blood through the filter in the vessel. While in some cases it is desirable to leave a medical filter in place indefinitely, in other cases it is desirable to retrieve the filter after it has been in place for a period of time. Such retrieval can be difficult. For example, when a vascular filter is placed within a vessel of the human body for any significant length of time, the inner lining of the vessel will grow around the portion of the device that is in contact with the tissue and encapsulate it. Tissue does not attach itself directly to materials such as Nitinol or stainless steel and it is known to make medical filters out of materials. However, depending upon the design of the filter, removal can still be difficult if there is mechanical interference between the tissue and elements of the filter. Corners, loops or other features of the filter must not interfere with tissue when the filter is retrieved in order for the retrieval to be atraumatic.

Another important feature desirable in a medical filter is that it be able to capture unwanted particles such as embolized blood clots without becoming occluded. It would also be desirable in some cases to have a vena cava or other medical filter designed to filter over a longer length than in a conventional filter to increase its clot capturing effectiveness while decreasing the risk of caval occlusion.

It is important that medical filters be resistant to migration. This is particularly the case with vascular filters. Thus, it is highly desirable that the filter resist movement in a vessel after it has been deployed yet be readily retrievable without tearing the tissue of the vessel. Of course, there are several other characteristics which are generally desirable in medical filters. For example, the filter should be of a design which can be deployed in a proper position in the blood vessel or body passage with minimal trauma to the patient and of a design which does not become entangled during its deployment. It is also desirable that the filter be adapted to center in the vessel and to properly adjust to the size of the vessel.

Although vascular and other medical filters are known in the art and have been found to be effective, the requirements of such filters can be high and there remains room for improved designs. In particular, there remains room for improved filters which exhibit the above-mentioned characteristics and yet can be economically manufactured. Accordingly, the present invention provides a medical filter which is suitable for placement in a blood vessel or body passage for an extended duration without migrating in the vessel or body passage and yet can be removed or retrieved with minimal trauma to the tissue of the blood vessel or body passage. Furthermore, the filter of this invention is effective for long duration and is resistant to occlusion.

In accordance with the present invention, a preferred embodiment of the present invention is an elongated medical filter which has a longitudinal axis, is radially expandable and compressible and is intended for placement inside a tubular body passage, such as a blood vessel, of a patient. In expanded form, the medical filter comprises:

(A) a longitudinally extending spine with a retrieval element at one end thereof;

(B) a plurality of filter arms spaced longitudinally on said spine in a helical pattern, each of arms extending generally radially outwardly and axially from said spine with a free end bent radially inwardly.

In one preferred embodiment of the present invention, the arms of each truncated filter basket are radially spaced at 60° radial angles to provide 6 arms per revolution. In another preferred embodiment the arms are spaced at about 30° radial angles to provide 12 arms per revolution. In a further alternative embodiment, each arm carries an additional element at the free end portion thereof to increase filtering efficiency.

Further understanding of the present invention will be had from the following description taken in conjunction with the accompanying drawings and appended claims.

### BRIEF DESCRIPTION OF THE DRAWINGS

FIG. 1 is a perspective view of a preferred embodiment of a medical filter of the present invention shown in expanded form;

FIG. 2 is a perspective view, broken away, showing preferred embodiment of the present invention having arms spaced at 30° intervals;

FIG. 3 is a perspective view, broken away, showing an alternative preferred embodiment of the present invention with arms at 60° intervals; and

FIG. 4 is an end view showing a preferred embodiment of the present invention having additional elements on the arms to provide increased filtering efficiency.

### DETAILED DESCRIPTION OF THE PREFERRED EMBODIMENTS

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the preferred embodiments of the present invention are described in conjunction with a blood vessel but the preferred embodiments may also be used in other body passages.

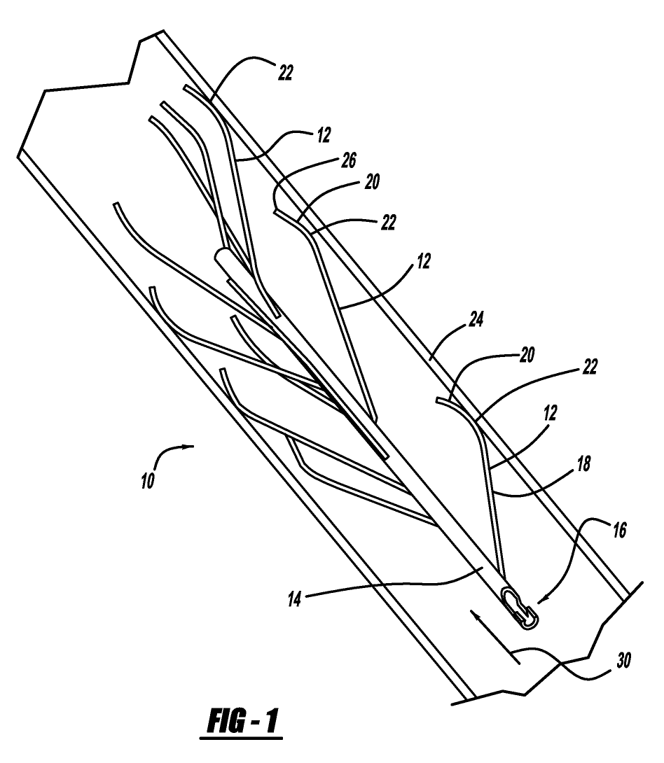

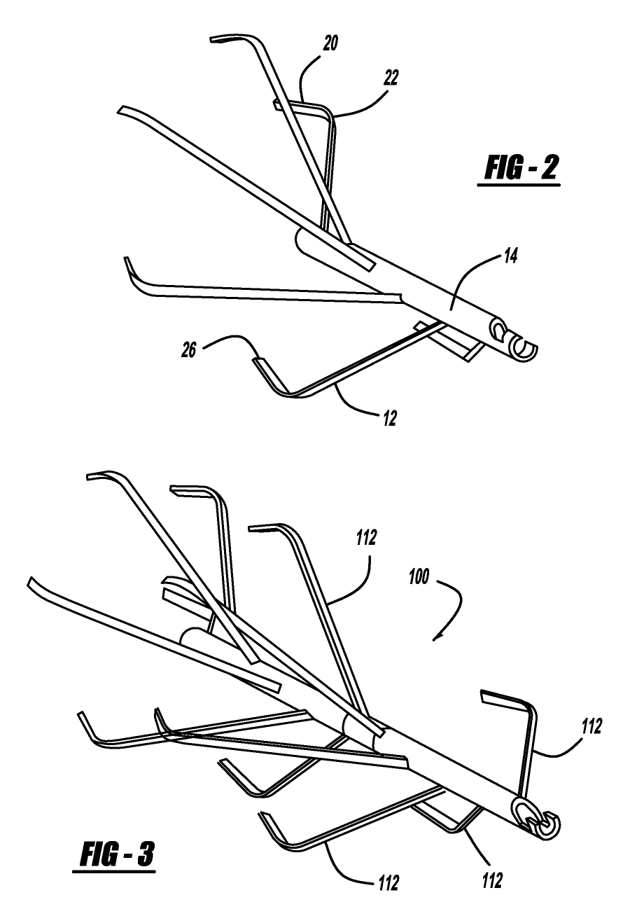

Now referring to FIGS. 1 and 2, a preferred embodiment of a medical filter of the present invention is shown and indicated generally by the numeral 10. Filter 10 is made of a resilient material, preferably a shape memory material, which tends to expand to the form illustrated in FIGS. 1 and 2 but can be compressed radially to a smaller diameter to be carried in the lumen of a suitable delivery catheter (not shown) as is conventional in the art.

Generally speaking medical filter 10 is radially expandable and compressible and is intended for placement inside a tubular body passage, such as a blood vessel, of a patient. It is contemplated that medical filter 10 will be compressed and placed into the lumen of a delivery catheter and then delivered to the desired site in a blood vessel or other body cavity whereupon filter 10 will be ejected from the catheter and allowed to self expand in position if the vessel or cavity. FIG. 1 illustrates filter 10 in expanded form in a blood vessel. In expanded form, medical filter 10 broadly comprises a plurality of arms 12 which are attached along spine 14 which has retrieval element 16 at one longitudinal end thereof.

Each arm 12 extends generally radially outwardly and axially from spine 14 and has a filter segment 18 and a reentrently bent end portion 20. Arm 12 thus has a bend 22 which comes into contact with an associated vessel wall 24 as illustrated in FIG. 1. Free end 26 of arm 12 is radially inward from vessel wall 24. Arms 12 are longitudinally spaced along spine 14 and extend from spine 14 in a helical manner. Thus, FIG. 2 illustrates arms 12 spaced radially each 60° about spine 14 to provide 6 arms for each “revolution” of spine 14.

Arms 12 can be formed as one piece with spine 14 or can be a separate piece attached to spine 14. Retrieval element 16 can be a retrieval hook or any other element adapted to facilitate retrieval of medical filter 10. Retrieval element 16 can be a single hook as shown in FIG. 1 or can have a T-shape with twin hooks or any other suitable shape which can operatively interact with a snare device or any other suitable element which will operatively interact with a retrieval means. It will be appreciated by those skilled in the art that hooks and snares are well-known and that the particular retrieval means employed is subject to variation within the spirit of the present invention.

Filter 10 may be made of any suitable material using a variety of methods. Nitinol is a preferred material but elgiloy, cobalt chromium, stainless steel or suitable plastic are examples of other materials that may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter. Thus, the material must also be sufficiently resilient to accomplish both compression in the delivery catheter and expansion upon ejection from the catheter.

Suitable methods of manufacture of medical filter 10 include cutting a pattern into a tube to enable expansion of the tube into the desired body and arms. Another suitable method is forming the arms and spine from separate strips or wires and then joining the respective parts together by suitable methods which are well known in the art.

Referring to FIG. 1, medical filter 10 is illustrated in use in blood vessel 24. The direction of flow of the blood stream is indicated by arrow 30. As is well illustrated in the FIG. 1, the blood flow 30 is substantially unimpeded by filter 10. Furthermore, any clots that bypass a filter leg are directed to the next available leg for entrapment. Thus, medical filter 10 has a low risk of occluding. It is contemplated that filter 10 will be preferably oriented with the arms extending generally downstream. Thus, clots or other unwanted thrombus or other material to be filtered will tend to be forced radially outwardly toward the wall of the blood vessel where they will tend to become trapped between an arm and the vessel wall.

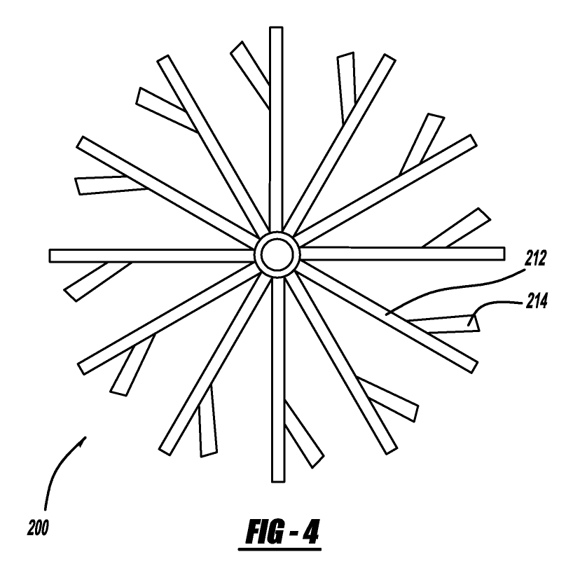

The exact number of arms of a filter of the present invention and the spacing thereof are subject to modification and variation within the spirit of the present invention. Thus, FIG. 3 illustrates an alternative preferred embodiment of the present invention, medical filter 100, in which the arms 112 are disposed at different radial locations about spine 130.

Referring to FIG. 4, another preferred embodiment of the present invention is shown and indicated generally by the numeral 200. Medical filter 200 is analogous to medical filter 100 except that each arm 212 has an extra element 214 on the end thereof for enhanced filtering efficiency.

The inward direction of free end segments 20 of arms 12 facilitate removal of filter 10 from an associated vessel or body passage. Thus, filter 10 has arms with no closed elements which will be in contact with a vessel wall and is thus designed for long term retrievability. Thus, although filter 10 is suitable for permanent placement in a vessel or body passage, filter 10 may be readily removed or retrieved if retrieval is desired. Retrieval of filter 10 may be accomplished by means of a conventional retrieval catheter which may be inserted from the appropriate direction to approach the end of filter 10 having retrieval element 16. After snaring retrieval element 16 with a snare, filter 10 is drawn into the lumen of the retrieval catheter and retrieved in a conventional manner. It will be appreciated that the free ends of the arms allow removal of the arms from any tissue that may have grown around them with minimal trauma.

It will be appreciated that the present invention offers several advantages over conventional medical filters. The present invention provides a filter that may be employed in any generally cylindrical pathway such as the vena cava of the human body. Any clot that approaches the filter will see a solid cylindrical impediment. However, the helical twist will allow the lower viscosity fluid medium to flow through and around the mesh. The clot will impinge the filter and either be trapped there, or be forced out toward the periphery by the helical centrifugal effect The structure of the filter will also induce the outward force on the filtrate by the inclination of the slanted arms, where the particulate will be trapped. The fluid is free to move around and past the clot, even if the filter structure is fully covered by the particulates. The present filter captures clots or other filtered material over a longer length that conventional filters and this increases the filter efficiency while decreasing the risk of caval occlusion. The filter can be placed above or across the renal veins in the inferior vena cava. The length of the filter offers increased resistance to migration after it is in place. The length and construction of the filter also makes it suitable for use in natural bends in the vena cava or other vessels.

While preferred embodiments of the present invention have been specifically described above, it will be appreciated by those skilled in the art that the present invention is subject to variations and modifications. which are contemplated to be within the scope of the present invention which is intended to be limited only by the following claims.

What is claimed is:

1. An elongated medical filter which has a longitudinal axis, is radially expandable and compressible and is intended for placement inside a tubular body passage, such as a blood vessel, of a patient. In expanded form, the medical filter comprises:
(A) a longitudinally extending spine with a retrieval element at one end thereof;
(B) a plurality of filter arms spaced longitudinally on said spine in a helical pattern, each of arms extending generally radially outwardly and axially from said spine with a free end bent radially inwardly.

2. A medical filter as in claim 1, wherein the arms of each truncated filter basket are radially spaced at 60° radial angles.

3. A medical filter as in claim 1, wherein the arms are spaced at about 30° radial angles.

4. A medical filter as in claim 1, wherein each arm carries an additional element at the free end portion thereof to increase filtering efficiency.

5. A medical filter as in claim 1, wherein said spine is comprised of a flexible material.

6. The medical filter of claim 1, wherein said retrieval element is a hook.

7. The medical filter of claim 1, wherein said filter is comprised of Nitinol.

8. The medical filter of claim 1 wherein said filter is comprised of stainless steel.

9. The medical filter of claim 1 wherein said arms are comprised of wire.

10. The medical filter of claim 1 wherein said arms are cut from tubing.

11. In combination with a vessel of a human body, an elongated medical filter comprising:
(A) a longitudinally extending spine with a retrieval element at one end thereof;
(B) a plurality of filter arms spaced longitudinally on said spine in a helical pattern, each of arms extending generally radially outwardly and axially from said spine with a free end bent radially inwardly.

12. A medical filter as in claim 11, wherein the arms of each truncated filter basket are radially spaced at 60° radial angles.

13. A medical filter as in claim 11, wherein the arms are spaced at about 30° radial angles.

14. A medical filter as in claim 11, wherein each arm carries an additional element at the free end portion thereof to increase filtering efficiency.

15. A medical filter as in claim 11, wherein said spine is comprised of a flexible material.

16. The medical filter of claim 11, wherein said retrieval element is a hook.

17. The medical filter of claim 11, wherein said filter is comprised of Nitinol.

18. The medical filter of claim 11 wherein said filter is comprised of stainless steel.

19. The medical filter of claim 11 wherein said arms are comprised of wire.

20. The medical filter of claim 11 wherein said arms are cut from tubing.
